(19) United States
(12) Patent Application Publication
(10) Pub. No.: US 2007/0207537 A1
(43) Pub. Date: Sep. 6, 2007

(54) Bioreactor

(76) Inventors: Zhanfeng Cui, Oxford (GB); Min-Hsein Wu, Kaohsiung City (TW); Xia Xu, Oxford (GB)

Correspondence Address: NIXON & VANDERHYE, PC, 901 NORTH GLEBE ROAD, 11TH FLOOR, ARLINGTON, VA, 22203, US

(21) Appl. No.: 11/725,546

(22) Filed: Mar. 20, 2007

Related U.S. Application Data
(63) Continuation of application No. PCT/GB05/03614, filed on Sep. 20, 2005.

(30) Foreign Application Priority Data
Sep. 20, 2004 (GB) 0420881.5

(51) Int. Cl. C12M 1/34; C12Q 1/20

(52) U.S. Cl. 435/288.4; 435/297.5; 435/33

(57) ABSTRACT

A cell culture plate for perfused cell or tissue culture comprises one or more wells. The wells are formed from a biocompatible gas permeable polymer. Means are provided to perfuse each well.

The present invention relates to a bioreactor, and in particular to apparatus carrying out cell culture. The apparatus is provided in particular for perfused cell and tissue culture.

### BACKGROUND TO THE INVENTION

Current cell and tissue culture systems essentially fall into two different categories, namely static culture using culture flasks and plates or perfused systems where nutrient or culture medium are continuously supplied to the cultured cells or tissues in a reactor. Static culture systems cannot maintain chemostat, particularly when Microsystems are used which may incorporate only a small volume of culture medium. In addition, there may be little control on the culture conditions used in static culture systems. However, static culture systems can be useful, in particular if small quantities of cells or tissue are to be cultured. Such systems typically use 96-well microtiter plates but larger or smaller plates may also be used.

The currently available perfused systems involve the use of large numbers of cells and cannot readily be scaled down to micro size. Thus, such perfused systems cannot be used to perform high throughput screens or parallel experiments in any economic way. Perfused systems can however reduce the risk of infection for long term cell/tissue culture which can occur with the regular medium change required when using static culture conditions.

### SUMMARY OF THE INVENTION

The present inventors have developed a system which allows for perfused culture of cells or tissues on a micro scale. In accordance with the present invention, there is provided a cell culture plate for perfused cell or tissue culture comprising one or more wells, wherein the wells are formed from a biocompatible gas permeable polymer, the plate further comprising means to perfuse each well.

### BRIEF DESCRIPTION OF THE DRAWINGS

FIG. 1 shows pH fluctuation in perfused microbiorector and static culture system (micro-well plate) in 6 day chondrocyte culture. The data represent averages “standard deviation for three independent experiments.

FIG. 2 shows glucose and lactate levels in perfused microbiorector and static culture system (micro-well plate) in 6 day chondrocyte culture. The data represent average values for three independent experiments.

FIG. 3 shows cell toxicity results using Alamar Blue, in cells cultured in the microbioreactor of the invention.

FIG. 4 shows the possible configuration of perfused membrane microbioreactors.

### DETAILED DESCRIPTION OF THE INVENTION

In accordance with the present invention, a plate or reactor comprising one or more wells or chambers for perfused cell or tissue culture is provided. Each well or chamber is formed from a biocompatible gas permeable polymer. Preferably the whole cell or tissue culture plate is made from the same polymer material. Fluid conduits are provided in each well for addition and removal of culture medium into each well. Preferably, the polymer allows for the introduction of fluid conduits such as needle like tubing which can simply be pushed through the polymer walls of the well. Preferably, the polymer is selected to form a seal around the conduits.

Typically, a plurality of wells are formed in each plate. Such wells may be formed into any suitable array. For example, an array of wells may be provided comprising 96 culture wells in a standard microtiter plate format. Such multi-well plates are preferred for ease of handling and also to allow for screening or processing of the plates using standard laboratory equipment. It will be appreciated that more or less wells could be provided in each plate. For example, the plate may hold between 1 and 1000 reaction wells, for example 24 to 384 reaction wells arranged in a suitable array. The plates may be configured to have any suitable dimension, depending on the size and number of reaction wells.

The volume of each well is preferably less than 10 ml, more preferably less than 5 ml, more preferably less than 2 ml. Typically, each well has a volume of around 1 ml or less (e.g. about 0.35 ml). Such wells may be used to culture tissue or cells in culture medium of a volume of, for example, 0.1 to 1.2 ml, preferably 0.2 to 1 ml. For a 96-well microplate, the dimensions are about 5 mm in inner diameter and about 18 mm in depth (total volume: about 0.35 ml). For a 48-well microplate, the dimensions are about 10 mm in inner diameter and about 17 mm in depth (total volume: about 1.3 ml). Cell and tissue culture can be performed with a small number of cells. Preferably, each well does not interconnect with any neighbouring wells to avoid any cross contamination of samples. Preferably part of the wall of each well is in contact with air or oxygen.

In some embodiments, means may be provided to allow different samples of cells to be in fluid contact with each other. Typically in this embodiment, a well or bioreactor chamber is provided with a membrane as described in more detail below. Alternatively two or more wells may be interconnected in particular to provide fluid connection between the wells. Such a system can be used for co-culturing of different cell types and/or to allow molecular markers secreted from cells in one well to be in contact with cells in the connected well.

Any suitable polymer material can be used to form the wells. Typically, the polymer is readily moulded in order to form the desired size and shape of well. Preferably the whole tissue or cell culture plate is made of the same polymer material, formed as a single cast.

The polymer is selected to be biocompatible, to avoid any adverse reaction with the cells or tissue to be cultured in the well. Preferably, the polymer is gas permeable. The polymer is typically permeable to O₂, CO₂ or both depending on the cells to be cultured. The gas permeability of the polymer can also be selected to control oxygen tension in each well.

The polymer is preferably an elastomer, to enable the polymer to form a seal around the fluid conduits inserted into each well. The polymer material is preferably a silicone polymer. Examples of preferred materials include polydimethylsiloxane (PDMS), polypropylmethylsiloxane (PPNS), polytrifluoropropylmethylsiloxane (PTFPMS), polyphenylmethylsiloxane (PPHMS).

The polymer is moulded or cast to produce any suitable well shape. Preferably, the wells have a circular or oval cross section. Alternatively, culture wells may have an elongated oval cross section. Square or rectangular shapes are not preferred since such shapes are not preferred for cell or tissue culture.

Preferably, the polymer is selected such that precursors including a curing agent can be poured into a mould and the mixture cured to produce the plate.

Each well is provided with fluid conduits (or connectors) to allow for the introduction and removal of fluid from the well. This allows for the supply of culture medium and removal of metabolic waste and/or spent medium from the cell culture. In a preferred embodiment, the fluid conduits are small diameter tubes such as needle-like tubes (e.g. biomedical-use needles) which can be inserted into each reaction well directly through a wall of each well. The tube can have a bevel tip. The reaction well may be provided with an adaptor to accommodate different tube sizes. The tube can be made from steel or other appropriate rigid material. The material is preferably non-toxic, and non-corrosive, and may be for example stainless steel. Generally, each fluid conduit has an outer diameter of from 0.3 to 6 mm, preferably from 0.6 to 3 mm. The inner diameter may be from 0.1 to 3 mm preferably from 0.3 to 1.5 mm.

The fluid conduits may be inserted through the base, side wall or, where present, the top wall of the well. The elastic properties of the polymer used may allow for self-sealing of the polymer around the inserted tubes.

Typically, each well or chamber is provided with two fluid conduits, one for introduction of culture medium and one for removal of spent medium. The fluid conduits may be provided in separate points in the well or chamber wall or may be provided adjacent to one another. Suitable pumping means are provided to allow circulation of fluid through the well. A multi-channel peristaltic or syringe pump is suitable. Each of the wells may be supplied by the same container of culture medium, with a single pump means provided to pump culture medium into each well. Suction means may be provided to assist in removal of medium for each well. Suitable control means can be used to provide uniform perfusion of the well, at selected rates of inflow and outflow. The flow rate depends on the capacity of the pump and the diameter of the tubing used. Typically, a flow rate of from 0.001 to 20 ml/hour may be used. Additional conduits may be provided for the addition of other components to the wells, such as the delivery of candidate compounds for analysis in the cell culture system. Alternatively, such agents may be delivered to the wells using the inflow fluid conduit or by direct introduction to the well opening.

Where the fluid conduits are provided for insertion through the base of each well, the conduits may be provided in a fixed array and the plate placed on top of the array of conduits to push each conduit through the polymer into the base of each well.

Preferably, each or the wells are provided with a cover. Typically, a cover is provided of the same polymer material as the plate to cover the or all of the reaction wells in the plate. However, the cover may be of a different material, and a hard cover is preferable. Where such a cover is present, the tubing for supply of culture medium and removal of spent medium or metabolic waste can be inserted through the cover.

The cover may be sealed to the plate. The plate and cover may effectively form the top and bottom parts of the reactor, or together form the chamber, the top and bottom parts being moulded into the desired shapes, and then brought together and sealed to create the bioreactor. The same polymer as used for the plate, or a biocompatible glue may be used to seal the two parts together. For example, a silicone gel may be used to seal the cover or the two parts of the reactor together. Silicone polymers such as PDMS, self-seal. In particular the clean surfaces of the top and bottom parts seal together when brought into contact with each other. The top and bottom parts may be provided with interlocking segments, such as a tongue and groove to facilitate sealing of the two parts together.

The polymer of the plate and/or the cover can be selected to be transparent. Such transparent wells or cover allow for direct observation of the cell/tissue culture, for example, under a microscope or using other techniques to analyse the cells such as fluorometry or spectrophotometry.

In a preferred embodiment, substantially all or all of the bioreactor, i.e. the plate and cover is made from the desired polymer, such as polysiloxane.

The plate can be used for cell or tissue culture, for example for the culture of chondrocytes. The number of cells cultured depends on the type of cell being cultured. For example, 10³ to 10⁸ cells, typically 10⁴ to 10⁶ cells, may be initially provided per well. In our experiments, about 2×10⁵ chondrocytes were initially provided per well and after 6-day culture there were 2.3×10⁵ cells per well. A scaffold or mesh can be provided in each well to assist in such cell or tissue culture where required. The duration of the culture is dependent on the cell type and the purpose of the culture being carried out. Typically, the culture is for 1 day to 1 year, for example 3 days to 6 months. For cartilage tissue culture, a maximum of about 6 weeks is typically needed.

In accordance with the present invention, perfused membrane microbioreactors may also be provided. Each microbioreactor in the system is made of biocompatible gas permeable polymer, such as PDMS, PPNS, PTFPMS and PPHMS. Preferably the whole cell/tissue culture system is made from the same polymer material including the cover of the microbioreactor.

The system has two parts, top and bottom part which may be identical. The type of two parts is the same as described before. The shape of each well in the system can be circular or oval in cross section although any suitable shape may be provided. The polymer is selected such that precursors including a curing agent can be poured into a mould and the mixture cured to produce the top and bottom plate. A membrane is placed in between the two parts. The membrane is sealed between these two parts using the same polymer or a biocompatible glue to achieve a permanent sealing. The membrane is preferably microporous typically with pore size ranging from 1 nm-100 nm (ultrafiltration membranes). Growth factor or protein secreted by cells during culture may be retained by a membrane of this pore size. For other purposes, for example to retain cells or support cell growth, microfiltration membrane with a pore size of 0.1 microns-20 microns can be used. Suitable materials for the membrane include polysulfone, polycarbonate, polyvinylidene fluoride (PVDF), regenerative cellulose, polyethersulfone, poly-lysine or other suitable material.

Each microbioreactor is provided with fluid conduits to allow for the introduction and removal of fluid from the microbioreactor. The distribution of the fluid conduits can be selected depending on the use. For example, both the top and the bottom part may have one fluid conduit, one for the introduction of culture medium, the other for the removal of waste from the well, or both the top and the bottom may have two fluid conduits to introduce and remove fluid from each part. The fluid conduits may be inserted through the top and the base wall of the well or side wall. See FIG. 4.

Any fluid delivery system, e.g. using a multi-channel peristaltic or syringe pump, is suitable for delivering and removing fluid from the well. Suitable control means can be used to provide uniform perfusion of the well at the selected rates of inflow and outflow. The flow rate is dependent on the capacity of the pump used. Additional conduits may be provided for the addition of other components to the wells.

### EXAMPLE 1

Materials and Methods

Fabrication of Microbioreactor

A plastic mould was machined by diamond milling to create 96, 48 well or smaller size of cylinder, which follows the standard 96- or 48-well plate. This mould was then used to cast silicone polymers such as polydimethylsiloxane (PDMS), polypropylmethylsiloxane (PPHMS), polytrifluoropropylmethylsiloxane (PTFPMS), polyphenylmethylsiloxane (PHMS). The mould was covered by the mixture of PDMS and its curing agent. The mixture was allowed to cure for overnight at 37° C. Once cured, the PDMS pattern was peeled off from the mould. The needle like tubing was introduced from the opposite sides for the nutrient supply and waste removal. Finally, the patterned PDMS was placed face-down onto a clean glass slide or a PDMS sheet to form microbioreactor. The microbiorector was autoclaved.

The connections with the external fluidic system were done by Pharmed® tubing. A culture medium bottle was connected to a peristaltic pump, which led to the microbioreactors.

Extraction of Chondrocytes from Bovine Feet

Bovine articular chondrocytes isolated from metacarpophalangeal joints were digested in modified Dulbecco's Modified Eagle's Medium (DMEM) supplemented with 1 mg/ml collagenase (Sigma type I), 1% (v/v) penicillin (10000 units/ml), streptomycin (10 mg/ml) and amphotericin (250 μg/ml) at 37° C./5% CO₂ for 18 h.

Preparation of Agarose Gel for Gel Imobilization and Perfusion Culture

4% agarose solution was made using PBS and autoclaved before use. A cell suspension with a cell density of 8 million cells/ml was mixed with 4% agarose gel at the ratio of 1:1. The mixture was transferred to the space between two plates to form a gel sheet with the thickness of 1 mm at 4° C. for 20 min. The gel was punched at the required size same as the microbioreactor, and then transferred to the microbioreactor. DMEM supplemented 6% Fetal Bovine Serum, antibiotics/antimycotics was used. The flow rate was around 0.0125 ml/h.

Results

Morphology and Cell Viability

The live/dead assay was used to do cell viability test. After six day culture, the cell-agarose construct was incubated in calcein AM solution (4 mM) and Ethidium homodimer-1 (EthD-1) solution (2 mM) at 37° C. for 20 min. The cell morphology in 4% agarose after six day culture using microbioreactor perfusion system was assessed. The cells keep the round shape. Nearly 100% of cells are alive after six day culture.

### EXAMPLE 2

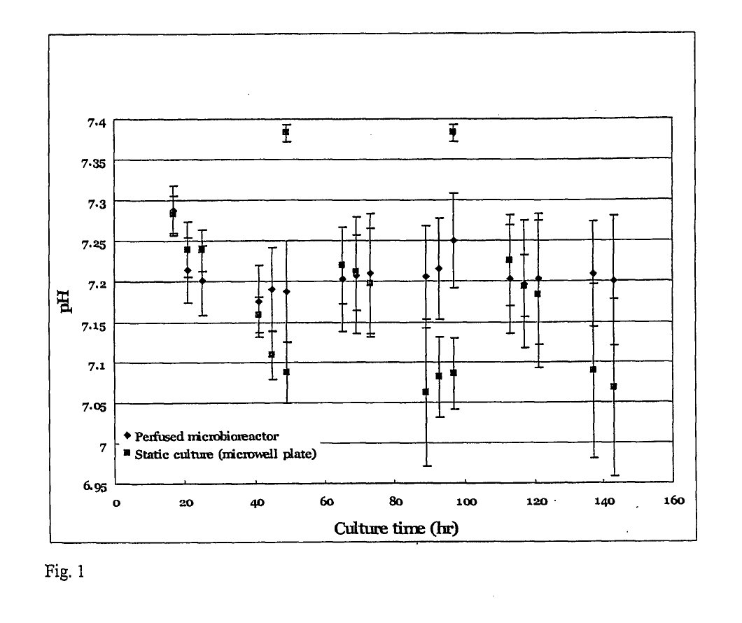

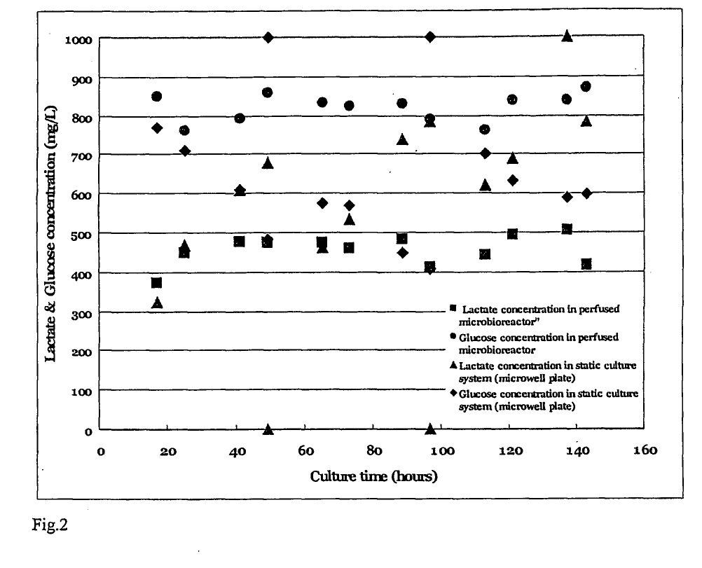

Further experiments were carried out to assess pH fluctuation and glucose and lactate fluctuation using the microbioreactor of Example 1 and in static culture. Bovine articular chondrocytes were isolated from metacarpa-phalangeal joints of 2-3 years old steers and embedded in 2% agarose gel disks of 7 mm diameter and 1.1 mm thickness at a cell density of 4×10⁶/ml. For the static system, each disk was cultured in one well of a 48 multi-well plate, containing 0.6 ml of sodium bicarbonate-free DMEM medium with 6% foetal calf serum, 2% antibiotics and antimycotics, and 50 μg/ml ascorbic acid at pH 7.4. The culture medium was changed every other day. For the perfusion system, the disk was cultured in a customized bioreactor with the flow rate of 12.5 μl/hr (an identical medium supply to that of the static system) for up to 6 days. A time course of pH fluctuation, lactate production and glucose consumption was obtained. The results are shown in FIGS. 1 and 2.

### EXAMPLE 3

Drug Testing or Chemical Toxicity Testing

The microbioreactors are fabricated following the procedure given in Example 1.

(1) Surface Modifications of Microbioreactor to Allow Cell Attachment

The sterilised poly-1-Lysine solution at the concentration of 0.01% (v/v) was introduced to each well of the microbioreactor system. After 12 h, the extra solution of poly-1-Lysine was removed from the microbioreactor. The microbioreactor was dried out overnight at room temperature in a sterilized environment.

(2) Preparation of Stem Cell Monolayer in the Microbioreactor and Perfusion Culture for Drug Testing

Human mesnchymal stem cells were suspended in the α Modified Eagle's Medium (MEM) at the cell density of 10⁴/ml. A 50 μl of cell suspension was introduced to each of the microbioreactors and kept at 37° C. After 4 h, the cell was seen to attach to the surface of the microbioreactor under microscope. During culture, αMEM supplemented with 15% FBS, 2% antibiotics/antimycotics and with different concentrations of tested drugs was used. The chemicals used were Trimethoprim and Pyrimethamine, which are known to be toxic to cells. The concentrations of the drug were 100 ng/ml and 250 ng/ml. The flow rate was 0.025 ml/h. Alamar Blue was used to do toxicity assay, which simply indicates the cell metabolity.

(3) Results

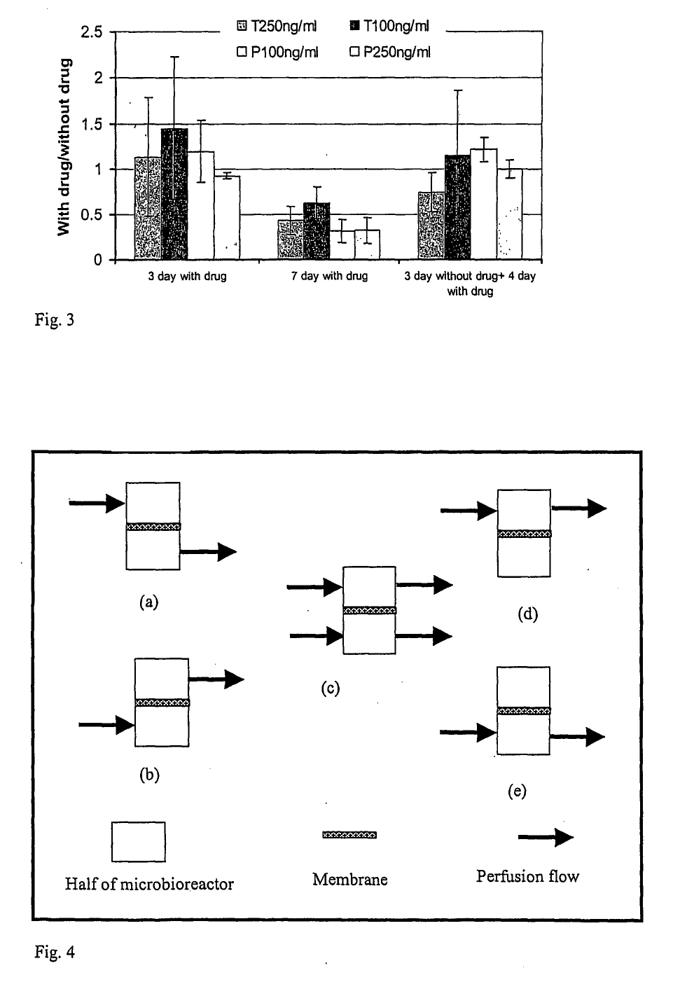

The samples from 3 days of culture with drug, 7 days of culture with drug and 3 days of culture without drug and further 4 days culture with drug were incubated with culture medium supplemented with Alamar Blue at day 3 and day 7, respectively for 4 h. Then 100 μl of culture medium was transferred to 96-well plate to read using microplate reader at the excitation wavelength of 530 nm and emission wavelength of 590 nm. Toxicity was shown as the ratio of reading from untreated with drug and from treated with drug, see FIG. 3. The toxicity was increased with culture time with drug. No significant different in toxicity between the two concentrations of drug in the test. However, a dramatic increase in toxicity was observed when 250 ng/ml of Pyrimethamine was used.

We claim:

1. A cell culture plate for perfused cell or tissue culture comprising one or more wells, wherein wells of the plate are formed from a biocompatible gas permeable polymer, and further comprising means to perfuse each well.

2. A plate according to claim 1, wherein the volume of each well is less than 10 ml.

3. A plate according to claim 2, wherein the volume of the or each well is less than 1 ml.

4. A plate according to claim 1 further comprising a plurality of wells.

5. A plate according to claim 4 having dimensions of a microtiter plate, wherein the plate is formed from the biocompatible polymer.

6. A plate according to claim 1, wherein the polymer comprises an elastomer.

7. A plate according to claim 6, wherein the polymer is a silicone polymer.

8. A plate according to claim 1, wherein the means to perfuse the well comprise one or more conduits for introduction and removal of fluid from the well, wherein the conduits are inserted through the polymer wall of each well.

9. A plate according to claim 1 further comprising a cover for the wells.

10. A plate according to claim 1, wherein the polymer is modified to improve biocompatibility.

11. A method of culturing a cell or tissue comprising incubating the cell or tissue in a well of a plate according to claim 1 and perfusing fluid through the well.

12. A cell culture chamber for perfused cell or tissue culture, wherein at least part of walls of the chamber are formed from a biocompatible gas permeable polymer and the chamber is provided with perfusion means, and further comprising a membrane dividing the chamber.

13. A chamber according to claim 12, wherein substantially all of the walls of the chamber are formed from a biocompatible gas permeable polymer.

14. A chamber according to claim 13, wherein the polymer is a silicone polymer.

15. A chamber according to claim 12, wherein the membrane is an ultrafiltration membrane having a pore size ranging from 1 to 100 nm.

16. A chamber according to claim 12, wherein the membrane is a microfiltration membrane having a pore size from 0.1 to 12 microns.

17. A chamber according to claim 12, wherein said perfusion means comprises one or more fluid conduits for introduction of fluid and one or more fluid conduits for removal of fluid from the chamber.

18. A method of testing the toxicity of a candidate compound comprising contacting the candidate compound with a cell or tissue cultured in a plate as defined in claim 1 or in a cell culture chamber, wherein at least part of walls of the chamber are formed from a biocompatible gas permeable polymer and the chamber is provided with perfusion means, and further comprising a membrane dividing the chamber.

19. A method according to claim 18, wherein an effect of the candidate compound is assessed by determining whether the candidate compound causes death or a reduction in metabolism of the cell or tissue.

20. A method according to claim 18, wherein the cell is a tumour or cancer cell, preferably of a human or mammal.
